(19) United States
(12) Patent Application Publication
(10) Pub. No.: US 2004/0224332 A1
(43) Pub. Date: Nov. 11, 2004

(54) System and method for calibration and focusing a scanner instrument using elements associated with a biological probe array

(75) Inventor: Gregory C. Loney, Concord, MA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/769,575

(22) Filed: Jan. 29, 2004

Related U.S. Application Data
(60) Provisional application No. 60/443,402, filed on Jan. 29, 2003.

(51) Int. Cl. C12Q 1/68; C12M 1/34

(52) U.S. Cl. 435/6; 435/287.2

(57) ABSTRACT

A system is described for providing focus elements associated with a biological probe array that comprises a biological probe array enabled to hybridize target molecules to probes disposed in an active area of the biological probe array; and focus elements disposed on the biological probe array, where the focus elements represent an unambiguous pattern.

RELATED APPLICATIONS

The present application claims priority to U.S. Provisional Patent Application Serial No. 60/443,402, titled “System, Method and Product providing Multiple Features for Automatic Scanner Focusing and Dynamic Image Analysis”, filed Jan. 29, 2003, which is hereby incorporated by reference herein in its entirety for all purposes. T he present invention is also related to U.S. patent application Ser. No. 10/389,194, titled “System, Method and Product for Scanning of Biological Materials”, filed Mar. 14, 2003, which is hereby incorporated by reference herein in its entirety for all purposes.

### FIELD OF THE INVENTION

The present invention relates to focus and calibration elements that enable scanning systems and software products to generate high quality data from biological probe arrays. In particular, the present invention relates to systems, methods, and products that include elements that enable a scanner and software applications to automatically place the probes of a probe array into the best plane of focus, and calibration elements that enables software applications to accurately measure and account for error present in a scanner instrument.

### BACKGROUND

Synthesized nucleic acid probe arrays, such as Affymetrix GeneChip® probe arrays, and spotted probe arrays, have been used to generate unprecedented amounts of information about biological systems. For example, the GeneChip® Human Genome U133 Plus 2.0 array available from Affymetrix, Inc. of Santa Clara, Calif., is comprised of a single microarray containing over 1,000,000 unique oligonucleotide features covering more than 47,000 transcripts that represent more than 33,000 human genes. Analysis of expression data from such microarrays may lead to the development of new drugs and new diagnostic tools.

### SUMMARY OF THE INVENTION

The expanding use of microarray technology is one of the forces driving the development of bioinformatics. In particular, microarrays and associated instrumentation and computer systems have been developed for rapid and large-scale collection of data about the expression of genes or expressed sequence tags (EST's) in tissue samples as well as data about the genotype of an individual.

Microarray technology and associated instrumentation and computer systems employ a variety of methods to obtain the accurate data from microarray experiments. Scanning of probe arrays is an essential step in microarray experiments and relies on their accurate placement within the scanning instruments; this in turn is of vital importance to obtain reliable data. Researchers are in need of increasingly accurate data generated by microarray technologies. A need exists to provide researchers with improved tools and methods to obtain increasingly accurate microarray experiment data in the minimum possible timeframe.

Systems, methods, and products are described herein to address these and other needs. Various alternatives, modifications and equivalents are possible.

A system is described for providing focus elements associated with a biological probe array that comprises a biological probe array enabled to hybridize target molecules to probes disposed in an active area of the biological probe array; and focus elements disposed on the biological probe array, where the focus elements include an unambiguous pattern.

In some embodiments, the focus elements are disposed outside of the active area, and include reflective elements such as chrome elements. In the same or alternative embodiments the focus elements are enabled to hybridize target molecules that may be present in a biological sample or added by a user. Also, the unambiguous pattern may include a checkerboard pattern.

Some implementations may also include, a scanner enabled to acquire an image of the focus elements; and an image analysis application enabled to execute one or more positional adjustments of the biological probe array based, at least in part, upon the image of the plurality of focus elements. Where the one or more positional adjustments of the biological probe array may include translating the biological probe array in one or more axes, such as the X, Y, Z, roll, and pitch axes, and may include placing each of the probes in a best plane of focus. In some embodiments, the image analysis application applies a deconvolution method to the image of the focus elements.

A method is described for positional adjustment of a biological probe array that comprises: acquiring an image of focus elements disposed on a biological probe array, where probes are disposed in an active area of the biological probe array; and executing on or more positional adjustments of the biological probe array based, at least in part, upon the image of the focus elements, where the focus elements include an unambiguous pattern.

In some embodiments, the focus elements are disposed outside of the active area, and the unambiguous pattern includes a checkerboard pattern. Also, the step of executing one or more positional adjustments includes translating the biological probe array in one or more axes, such as X, Y Z, roll, and pitch axes that may include placing each of the probes in a best plane of focus.

A method is described for positional adjustment of a biological probe array that comprises: acquiring an image of focus elements disposed on a biological probe array, where probes are disposed in an active area of the biological probe array; applying a deconvolution method to the image of the focus elements; and executing one or more positional adjustments of the biological probe array based, at least in part, upon the deconvolution method of the image.

In some embodiments, the step of executing one or more positional adjustments includes placing each of the plurality of probes in a best plane of focus.

A system is described for providing calibration elements associated with a biological probe array that comprises: a biological probe array enabled to hybridize target molecules to probes disposed in an active area of the biological probe array; and calibration elements disposed on the biological probe array, where the calibration elements are disposed in a rectilinear pattern.

In some embodiments, the calibration elements are disposed in the active are of the biological probe array and may include a chrome element. Also, each of the calibration elements may include a vertical component and a horizontal component, where the vertical component may be associated with X-axis linearity and the horizontal component may be associated with Y-axis linearity.

Some implementations may also include a scanner enabled to acquire an image of the target molecules hybridized to the probes and the calibration elements; and an image analysis application enabled to generate a plurality of error correction values. In some embodiments, each error correction value may be based, at least in part, upon a difference between a position of each calibration element in the acquired image and an actual position of a corresponding calibration element disposed on the biological probe array, where each error correction value may be associated with a pixel position in the image.

A method is described for determining error associated with a scanner instrument that comprises: acquiring an image of target molecules hybridized to a plurality of probes and a plurality of calibration elements; and generating error correction values, where each error correction value is based, at least in part, upon a difference between a position of each calibration element in the acquired image and an actual position of a corresponding calibration element disposed on the biological probe array.

In some embodiments, the calibration elements are disposed in a rectilinear pattern in the active are of the biological probe array.

A method is described for determining error associated with a scanner instrument that comprises: acquiring an image of target molecules hybridized to a plurality of probes and a plurality of calibration elements; generating error correction values, where each error correction value is based, at least in part, upon a difference between a position of each calibration element in the acquired image and an actual position of a corresponding calibration element disposed on the biological probe array; and correlating one or more of the error correction values with each pixel in the image, where the correlating includes adjusting the position of each pixel based, at least in part, upon the one or more error correction values.

In some embodiments, the error correction value includes an X-axis linearity correction value, a Y-axis linearity correction value or both.

A system is described for providing focus elements associated with a plurality biological probe arrays that comprises: a plurality of biological probe arrays each enabled to hybridize target molecules to probes disposed in an active area of each biological probe array; and focus elements disposed on the biological probe array, where the focus elements include an unambiguous pattern.

A system is described for providing calibration elements associated with a plurality biological probe arrays that comprises: a plurality of biological probe arrays each enabled to hybridize target molecules to probes disposed in an active area of each biological probe array; and calibration elements disposed on the biological probe array, where the calibration elements are disposed in a rectilinear pattern.

The above implementations are not necessarily inclusive or exclusive of each other and may be combined in any manner that is non-conflicting and otherwise possible, whether they be presented in association with a same, or a different, aspect or implementation. The description of one implementation is not intended to be limiting with respect to other implementations. Also, any one or more function, step, operation, or technique described elsewhere in this specification may, in alternative implementations, be combined with any one or more function, step, operation, or technique described in the summary. Thus, the above implementations are illustrative rather than limiting.

### BRIEF DESCRIPTION OF THE DRAWINGS

The above and further advantages will be more clearly appreciated from the following detailed description when taken in conjunction with the accompanying drawings. In the drawings, like reference numerals indicate like structures or method steps and the leftmost one or two digits of a reference numeral indicate the number of the figure in which the referenced element first appears (for example, element 100 appears first in FIG. 1). In functional block diagrams, rectangles generally indicate functional elements, parallelograms generally indicate data, rectangles with curved sides generally indicate stored data, rectangles with a pair of double borders generally indicate predefined functional elements, and keystone shapes generally indicate manual operations. In method flow charts, rectangles generally indicate method steps and diamond shapes generally indicate decision elements. All of these conventions, however, are intended to be typical or illustrative, rather than limiting.

FIG. 1 is a functional block diagram of one embodiment of a probe array, scanner and computer system that includes an instrument control and image processing application;

FIG. 2A is a simplified graphical illustration of one embodiment of the probe array of FIG. 1 with a plurality of elements disposed in a region outside the active area of the probe array, where the elements are enabled for use in focusing operations;

FIG. 2B is a simplified graphical illustration of one embodiment of the element of FIG. 2A having a pattern recognizable by image analysis software;

FIG. 3A is a simplified graphical illustration of one embodiment of the probe array of FIG. 1 with a plurality of calibration elements disposed thereon;

FIG. 3B is a simplified graphical illustration of one embodiment of the calibration elements of FIG. 3A spatially positioned with respect to a plurality of probe features;

FIG. 4 is a functional block diagram of one embodiment of the instrument control and image processing application of FIG. 1 that includes a pattern recognition filter and a focus correction generator;

FIG. 5 is a functional block diagram of one embodiment of the instrument control and image processing application of FIG. 1 that includes a calibration data generator and a image correction correlator; and

FIG. 6 is a functional block diagram of one embodiment of a method for generating a plurality of error correction values.

### DETAILED DESCRIPTION

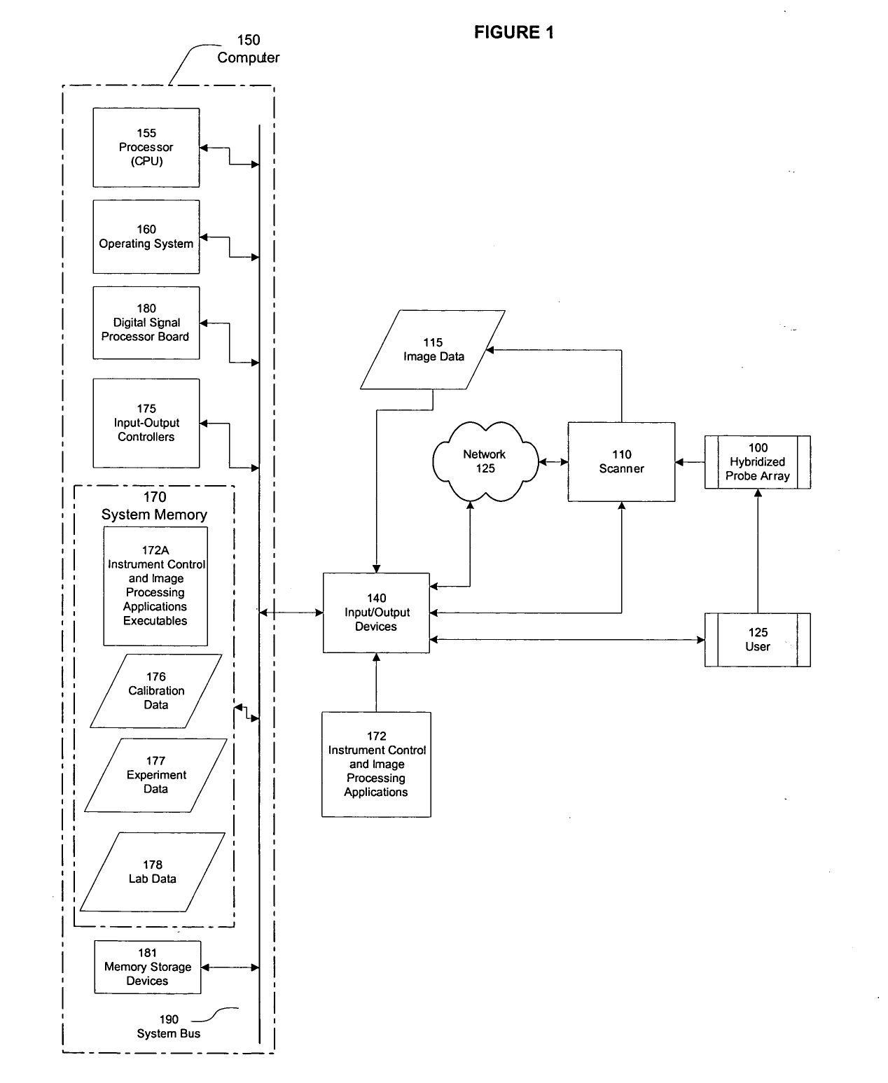

The present invention may be embodied as a system for calibration and/or focusing using one or more elements associated with a biological probe array, a method, of data processing and/or handling system, computer software program product or products, or any combination thereof. Illustrative embodiments are now described with reference to the probe array and computer system as illustrated in FIG. 1. The operations of this computer system and of instrument control and image processing applications executables 172A such as, for instance, the GCOS software application that are executed on computers of this system, are illustrated in the context of generating, processing, and handling of data generated from hybridized probe arrays, such as arrays 100 of FIG. 1. This data generating includes the scanning of arrays 100 by scanner 110 and the processing of the resulting information (and other data) by software executing on representative computer 150 such as the instrument control and image processing applications executables 172A. Further, data handling and other aspects of management is carried out by the image processing applications executables 172A enabled to utilize local and remote resources such as available on a server.

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285 (International Publication Number WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598 (U.S. patent application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H.A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and W088/10315), self-sustained sequence replication (Guatelli et al., Proc. Nat. Acad. Sci. USA, 87, 1874 (1990) and W090/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. U.S. 20020183936), 10/065,856, 10/065,868, 10/328,818, 10/328,872, 10/423,403, and 60/482,389.

b) Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Altematively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about 1M, more usually less than about 500 mM and preferably less than about 200 mM. Hybridization temperatures can be as low as 5.degree. C., but are typically greater than 22.degree. C., more typically greater than about 30.degree. C., and preferably in excess of about 37.degree. C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25 degrees-Celsius (° C.), e.g., 750 mM NaCl, 50 mM NaPhosphate, 5 mM EDTA, pH 7.4 (5×SSPE)and a temperature of from about 25 to about 30° C.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2nd Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (MRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

MRNA or MRNA transcripts: as used herein, include, but not limited to pre-mRNA transcript(s), transcript processing intermediates, mature mRNA(s) ready for translation and transcripts of the gene or genes, or nucleic acids derived from the MRNA transcript(s). Transcript processing may include splicing, editing and degradation. As used herein, a nucleic acid derived from an MRNA transcript refers to a nucleic acid for whose synthesis the MRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, MRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety. “Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

c) Embodiments of the Present Invention

Hybridized Probe Arrays 100: Illustrated in FIG. 1 is Hybridized Probe Arrays 100. Various techniques and technologies may be used for synthesizing dense arrays of biological materials on or in a substrate or support as described above that may then be exposed to biological samples containing a plurality of target molecules that specifically hybridize to probes disposed upon the array. Those of ordinary skill in the related art will appreciate that probe arrays may be produced using a variety of substrates that may comprise a variety of shapes and sizes. For example, probe arrays may be produced on solid, semi-rigid, or flexible substrates that also may be planar, spherical, comprise a pattern of repeating elements, or some other non-uniform shape. Also, a planar substrate may be elongate such as a tape or ribbon, square, rectangular, circular, or other shape known to those of ordinary skill.

In some embodiments, one or more methods or steps of processing biological probe array or image acquisition may be carried out by one or more instruments such as, for instance, what may be referred to as a fluidics station, scanner 110 or other type of instrument.

Scanner 110: Labeled targets hybridized to probe arrays 100 may be detected using various devices, sometimes referred to as scanners, as described above with respect to methods and apparatus for signal detection. An illustrative device is shown in FIG. 1 as scanner 110. For example, scanners image the targets by detecting fluorescent or other emissions from labels associated with target molecules, or by detecting transmitted, reflected, or scattered radiation. A typical scheme employs optical and other elements to provide excitation light and to selectively collect the emissions.

Those of ordinary skill in the related art will appreciate that scanner instruments such as scanner 110 may include one or more of a variety of different types of components that may accomplish the same purpose. For example, sources of excitation light may include one or more lasers, wide spectrum sources such as, for instance, Xenon arc type bulbs, Light Emitting Diodes, or other type of excitation source known to those of ordinary skill. Similarly, detectors of emissions and other wavelengths of light may include CCD cameras, cooled CCD cameras, Photo-Multiplier Tubes, Super-conductor based light detectors, Photo Diodes, and other light detectors known to those of ordinary skill.

For example, scanner 110 provides image data 115 representing the intensities (and possibly other characteristics, such as color) of the detected emissions or reflected wavelengths of light, as well as the locations on the substrate where the emissions or reflected wavelengths were detected. Typically, image data 15 includes intensity and location information corresponding to elemental sub-areas of the scanned substrate. The term “elemental” in this context means that the intensities, and/or other characteristics, of the emissions or reflected wavelengths from this area each are represented by a single value. When displayed as an image for viewing or processing, elemental picture elements, or pixels, often represent this information. Thus, in the present example, a pixel may have a single value representing the intensity of the elemental sub-area of the substrate from which the emissions or reflected wavelengths were scanned. The pixel may also have another value representing another characteristic, such as color, positive or negative image, or other type of image representation. Two examples of image data 115 are data files in the form *.dat or *.tif as generated respectively by Affymetrix® Microarray Suite (described in U.S. patent application Ser. No. 10/219,882, incorporated above) or Affymetrix GeneChip® Operating Software based on images scanned from GeneChip® arrays, and Affymetrix® Jaguar™ software (described in U.S. Provisional Patent Application No. 60/226,999, incorporated above) based on images scanned from spotted arrays.

Computer 150: Image data 115 may be stored and/or processed by a computer system such as any one or more of a number of computers connected to network 125, generally and collectively referred to as computer 150. In alternative implementations, image data 276 may be provided by computer 150, via network 125, to a server (not shown) where it may similarly be stored and/or processed. An example of computer 150 is illustrated in FIG. 1. Computer 150 may be any type of computer platform such as a workstation, a personal computer, a server, or any other present or future computer. Computer 150 typically includes known components such as a processor 155, an operating system 160, a system memory 170, memory storage devices 181, input-output controllers 175, and input/output devices 140. In particular, output controllers of input-output controllers 175 could include controllers for any of a variety of known display devices, network cards, and other devices well known to those of ordinary skill in the relevant art. Input/Output Devices 140 may include display devices that provides visual information, this information typically may be logically and/or physically organized as an array of pixels. A Graphical user interface (GUI) controller may also be included that may comprise any of a variety of known or future software programs for providing graphical input and output interfaces to a user, such as user 125, and for processing user inputs.

Instrument control and image processing applications 172: Instrument control and image processing applications 172 may be any of a variety of known or future image processing applications. Examples of applications 172 include Affymetrix® Microarray Suite, Affymetrix® GeneChip® Operating Software (hereafter referred to as GCOS), and Affymetrix® Jaguar™ software, noted above. Applications 172 may be loaded into system memory 170 and/or memory storage device 181 through one of input devices 140. Applications 172 as loaded into system memory 170 are shown in FIG. 1 as instrument control and image processing applications executables 172A.

Embodiments of applications 172 includes executables 172A being stored in system memory 170 of an implementation of computer 150 that includes what is commonly referred to by those of ordinary skill in the related art as a client workstation. Executables 172A may provide a single interface for both the client workstation and one or more servers such as, for instance, GeneChip® Operating Software Server (GCOS Server). Executables 172A could additionally provide the single user interface for one or more other workstations and/or one or more instruments. In the presently described implementation, the single interface may communicate with and control one or more elements of the one or more servers, one or more workstations, and the one or more instruments. In the described implementation the client workstation could be located locally or remotely to the one or more servers and/or one or more other workstations, and/or one or more instruments. The single interface may, in the present implementation, include an interactive graphical user interface that allows a user to make selections based upon information presented in the GUI.

In alternative implementations, applications 172 may be executed on a server, or on one or more other computer platforms connected directly or indirectly (e.g., via another network, including the Internet or an intranet) to network 125.

Embodiments of applications 172 also include instrument control features. The instrument control features may include the control of one or more elements of one or more instruments that could, for instance, include elements of a fluidics station, what may be referred to as an autoloader, and scanner 110. The instrument control features may also be capable of receiving information from the one more instruments that could include experiment or instrument status, process steps, or other relevant information. The instrument control features could, for example, be under the control of or an element of the single interface. In the present example, a user may input desired control commands and/or receive the instrument control information via a GUI.

In the illustrated embodiment of FIG. 1, image data 115 is operated upon by executables 172A to generate intermediate results. Examples of intermediate results include so-called cell intensity files (*.cel) and chip files (*.chp) generated by Affymetrix® GeneChip® Operating Software or Affymetrix® Microarray Suite (as described, for example, in U.S. patent application, Ser. No. 10/219,882, hereby incorporated herein by reference in its entirety for all purposes) and spot files (*.spt) generated by Affymetrix® Jaguar™ software (as described, for example, in PCT Application PCT/US 01/26390 and in U.S. patent applications, Ser. Nos. 09/681,819, 09/682,071, 09/682,074, and 09/682,076, all of which are hereby incorporated by reference herein in their entireties for all purposes). For convenience, the term “file” often is used herein to refer to data generated or used by executables 172A and executable counterparts of other applications, but any of a variety of alternative techniques known in the relevant art for storing, conveying, and/or manipulating data may be employed.

In one of the examples noted above, executables 172A receives image data 115 derived from a GeneChip® probe array and generates a cell intensity file. This file contains, for each probe scanned by scanner 110, a single value representative of the intensities of pixels measured by scanner 110 for that probe. Thus, this value is a measure of the abundance of tagged mRNA's present in the target that hybridized to the corresponding probe. Many such mRNA's may be present in each probe, as a probe on a GeneChip® probe array may include, for example, millions of oligonucleotides designed to detect the mRNA's. As noted, another file illustratively assumed to be generated by executables 172A is a chip file. In the present example, in which executables 172A include Affymetrix® GeneChip® Operating Software, the chip file is derived from analysis of the cell file combined in some cases with information derived from lab data and/or library files that specify details regarding the sequences and locations of probes and controls. The resulting data stored in the chip file includes degrees of hybridization, absolute and/or differential (over two or more experiments) expression, genotype comparisons, detection of polymorphisms and mutations, and other analytical results.

In another example, in which executables 172A includes Affymetrix® Jaguar™ software operating on image data from a spotted probe array, the resulting spot file includes the intensities of labeled targets that hybridized to probes in the array. Further details regarding cell files, chip files, and spot files are provided in U.S. patent application Ser. Nos. 09/682,098, 09/682,071, and 10/126,468, incorporated by reference above. As will be appreciated by those skilled in the relevant art, the preceding and following descriptions of files generated by executables 172A are exemplary only, and the data described, and other data, may be processed, combined, arranged, and/or presented in many other ways.

User 125 and/or automated data input devices or programs (not shown) may provide data related to the design or conduct of experiments. As one further non-limiting example related to the processing of an Affymetrix® GeneChip® probe array, the user may specify an Affymetrix catalogue or custom chip type (e.g., Human Genome U95Av2 chip) either by selecting from a predetermined list presented by GCOS or by scanning a bar code related to a chip to read its type. GCOS may associate the chip type with various scanning parameters stored in data tables including the area of the chip that is to be scanned, the location of chrome borders on the chip used for auto-focusing, the wavelength or intensity of laser light to be used in reading the chip, and so on. These and other data are represented in FIGS. 1 as lab data 178. Data 178 may include, for example, the name of the experimenter, the dates on which various experiments were conducted, the equipment used, the types of fluorescent dyes used as labels, protocols followed, and numerous other attributes of experiments. As noted, executables 172A may apply some of this data in the generation of intermediate results. For example, information about the dyes may be incorporated into determinations of relative expression. An additional example of systems and methods enabled for instrument control and image processing such as, for instance those employed by the GCOS software application, are provided in U.S. Patent Application Attorney Docket No. 3348.9, titled “System, Method and Computer Software Product for Instrument Control, Data Acquisition, Analysis, Management and Storage”, filed Jan. 26, 2004, which is hereby incorporated by reference herein in its entirety for all purposes.

In some embodiments of the present invention, executables 172A may use image data 115 that may for instance include a * .dat or *.tif file, for focusing or calibration methods that will be discussed in greater detail below with respect to auto-focus operations and calibration of scanner 110.

Auto-Focus Operation: Some embodiments of scanner 110 employ one or more methods of automatically bringing the features of probe array 100 into the plane of best focus that is substantially normal to the plane of an excitation beam. Such methods do not require the intervention by user 125, and may employ one or more elements associated with probe array 100, where the elements are preferably disposed in substantially the same plane as the substrate and/or features of probe array 100. For example, some embodiments include a chrome border disposed in a region surrounding what may be referred to as the “active area” of probe array 100 where the plane of best focus may be defined by the plane at which a spot produced by an excitation beam produced by scanner 110 makes the quickest transition across the edge of the chrome border, i.e. substantially the smallest spot size produced by what is referred to as a convergent/divergent beam. One or more transport elements may be employed by scanner 110 enabled to accurately position probe array 100 in multiple axes such as, for instance, the roll, pitch, X, Y, and Z (focus) axes. Further examples of employing a chrome border in auto focus methods as well as related embodiments of probe array transport and position control is described in U.S. patent application Ser. No. 10/389,194, incorporated by reference above.

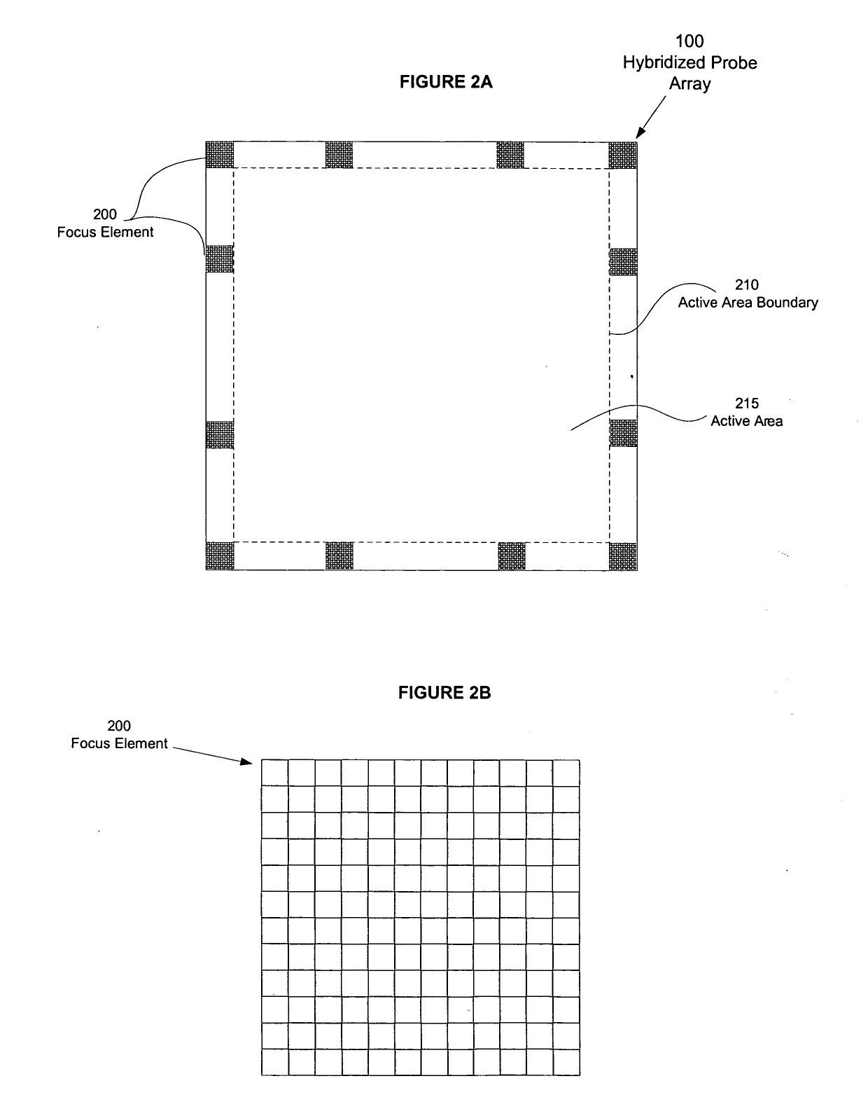

FIGS. 2A and 2B provide an illustrative example of one possible implementation of elements associated with probe array 100 that may be employed by executables 172A as a recognizable feature to determine the best plane of focus. Some embodiments may include elements such as focus element 200 that may be disposed upon probe array 100 or alternatively disposed on a substrate such as, for instance, a transparent cover over probe array 100 where the transparent cover is substantially in the same plane as probe array 100. As illustrated in FIG. 2A, embodiments of focus elements 200 may be disposed on probe array outside of what may be referred to as the active area such as, for instance active area 215 defined by active area boundary 210. The term “active area” as used herein generally refers to the region or regions of a biological probe array occupied by one or more biological probes or probe sets. For example, one or more of elements 200 may be positioned at one or more locations on probe array 100 such as, for instance at the comers, and/or along one or more edges as illustrated in FIG. 2A. In the present example, having elements 200 disposed outside of active area 215 enables executables 172A to execute multiple iterations of exposing elements 200 to excitation light in order to determine the distance between an objective lens and probe array 100 associated with the best plane of focus, without substantial risk of what may be referred to by those of ordinary skill in the related art as photobleaching of fluorescent molecules associated with target molecules that have hybridized to probes of probe array 100.

In some embodiments the best plane of focus may be defined by a predetermined size of a spot of an excitation beam focused onto probe array 100 and/or focus elements 200. Some embodiments of focus elements 200 may include chrome or other type of reflective material to define a specific pattern where the spot or excitation beam is reflected back to the scanner optics and detectors. For example, executables 172A may recognize reflected excitation light as a positive or negative image with respect to emitted light collected from fluorescent molecules.

Embodiments of the present invention may include elements 200 comprising at least one type of probe molecule that selectively bind to a target molecule present in a biological sample or added as part of an experimental protocol by user 125. For example, at least one type of probe molecule may be disposed upon probe array 100 in a recognizable and unambiguous pattern such as, for instance, the checkerboard type pattern of FIG. 2B. In the present example, the recognizable pattern may be consistent for all of elements 200 disposed upon array 100, or alternatively a particular pattern may be specific to a particular region of probe array 100, where executables 172A may use each particular pattern to define one or more spatial relationships associated with probe array 100. Continuing with the present example, patterns may include alternating horizontal or vertical lines, concentric circles or a combination of other shapes and patterns known to those of ordinary skill in the related art.

Some examples of scanning probe array 100 for automatic focusing operations include an objective lens that focuses an excitation beam to a point and scans along an arc in the direction of what may be referred to as the X-axis or “fast scanning” axis. The excitation beam may be reflected from or excite one or more fluorescent markers associated with target molecules, the light collected by the scanner optics and detectors, and translated into image data such as image data 115. In some embodiments, probe array 100 may then be translated some distance along what may be referred to as the Y axis or “slow scanning” axis and the X-axis scan performed again. Alternatively, the distance between an objective lens and probe array 100 may be adjusted by executables 172A and the X-axis scan repeated. In the present example, multiple iterations of scanning along a plurality of positions on the Y axis employing a plurality of lens-array distances may be employed to generate enough image data for executables 172A to make an accurate determination of the best plane of focus for substantially all points on probe array 100. Also in the present example, executables 172A may adjust the position of probe array 100 in the Z, pitch and roll axes individually or in a variety of combinations to achieve best focus.

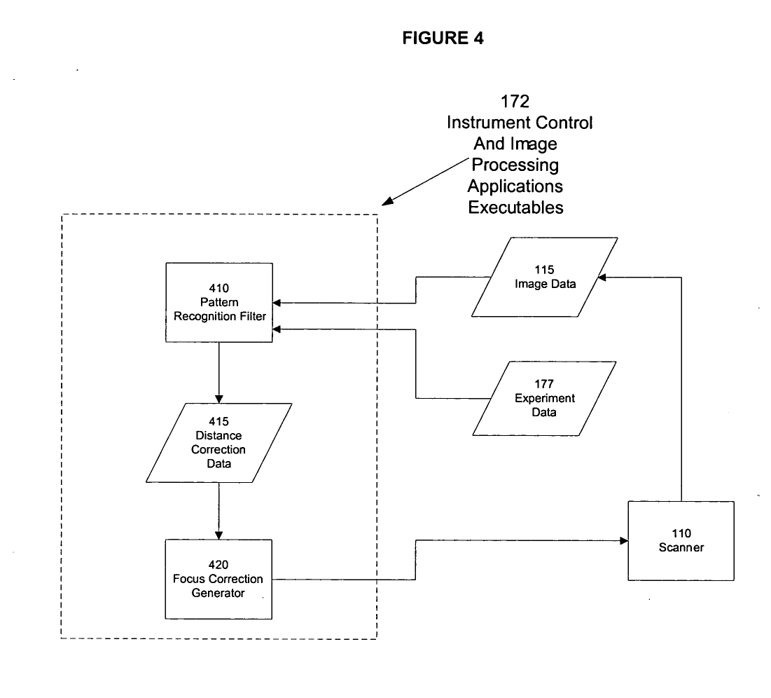

Embodiments of the present invention may include executables 172A employing a means of pattern recognition whereby, executables 172A is enabled to identify the best plane of focus in a single pass of an excitation beam over elements 200, thus eliminating the need for multiple iterations. For example as illustrated in FIG. 4, an component of executables 172A such as pattern recognition filter 410 may receive image data 115 from scanner 110 that corresponds to a single pass of an excitation beam over a plurality of elements 200, and pattern data from experiment data 177. Pattern recognition filter 410 employs one or more pattern matching algorithms and/or deconvolution algorithms known to those of ordinary skill in the related art to determine one or more values associated with a degree of error based, at least in part, upon the relationship of the algorithm results to the actual unambiguous pattern received from experiment data 177. In the present example, the degree of error is proportional to one or more distance correction values, illustrated as distance correction data 415, that may be employed by executables 172A to adjust the distance between the objective lens and probe array 100 so that substantially all of the points of probe array 100 are in the best plane of focus. Continuing with the present example, distance correction data 415 is employed by focus correction generator 420 to instruct scanner 110 to translate probe array 100 in one or more axes, such as the Z, roll, pitch axes.

Additionally, some embodiments of executables 172A may employ a number of variables to perform its automatic focusing operations. For example, one of the many possible variables may include one or more probe array type variables that define one or more physical parameters necessary for accurate positioning of probe array 100 in the best plane of focus. In the present example the one or more probe array type variables may be downloaded via network 125, loaded from removable storage such as a CD, or otherwise provided by user 125 and stored in a data file or database such as experiment data 177.

Some embodiments of elements 200 may be associated with a plurality of probe arrays 100, such as for instance a plurality of probe arrays 100 disposed upon a wafer or separated into individual wells etc. For example, elements 200 may be disposed outside of the active area of each individual probe array 100, where the elements 200 may define a boundary area between each probe array 100 embodiment such as on a wafer implementation. In the present example, executables 172A may perform focus operations for each implementation of probe array 100 disposed on a wafer or in individual wells based, at least in part, upon one or more of the focus elements 200 associated with the implementation of probe array 100.

Also, some embodiments of focus elements may be disposed in the active area of probe array 100 such as, for instance, within active area boundary 210. For example, scanner 110 may include two sources of excitation light such as, for instance, two lasers where the lasers emit excitation beams of different wavelengths, a first laser may emit a beam that is outside the excitation spectra of the fluorescent molecules associated with the hybridized experimental probes where the first laser may excite fluorescent molecules associated with hybridized focus element probes that for instance whose targets are unique from the experimental targets. The second laser may emit a beam that excites the fluorescent molecules associated with the hybridized experimental probes where the laser may be used serially or in tandem. In the present example, the first laser may be employed to scan probe array 100 and allow executables 172A to perform automatic focusing operations without risk of photobleaching the fluorescent molecules associated with the hybridized experimental probes. Those of ordinary skill in the related art will appreciate that placing elements 200 in the active area may be especially useful with non- uniform, semi-rigid, or flexible substrates where executables 172A may dynamically place a particular area of probe array 100 of interest in the best plane of focus.

Yet another embodiment of focus elements 200 may be implemented using CCD or cooled CCD type detectors where an image may be acquired and applied to a discrimination function or other type of algorithm that may be employed by a machine to recognize a pattern.

Those of ordinary skill in the related art will appreciate that each of focus elements 200 may be employed for one or other methods of instrument calibration such as, for example, what may be referred to as gain calibration of one or more detector elements of scanner 110 that could be used to reduce what is referred to as “noise” associated with image data 115.

Calibration of Scanner 110: It may be desirable in many implementations to calibrate one or more components of scanner 110 such as, for instance, to compensate for error caused by variation in the components that could affect the quality of image data 115. Such variations could include those caused by mechanical or optical elements that are affected by a variety of factors including temperature fluctuation, mechanical wear, component imperfections, slight changes caused by physical insult, or other of a variety of possible factors. In one implementation, scanner 110 could be initially calibrated at the factory and/or during routine service in the field. Alternatively or in addition to the previous implementation, one or more calibration operations could be automatically performed prior to or during each scan, during the power-up routine of the scanner, or at other frequent intervals. For example, scanner 110 may be calibrated with respect to one or more spatial characteristics of mechanical movement as well as optical characteristics that include calibration of light collection components and radius of the light path calculations as described in U.S. patent application Ser. No. 10/389,194, incorporated by reference above. Those of ordinary skill in the related art appreciate that frequent calibration of sensitive components enable scanner 110 to provide the most accurate images, and preferably would not require intervention from a user to perform the calibration procedures.

In some embodiments of probe array 100, individual probe features may approach very small sizes such as, for instance, square probe features in the range of 1-5 μm in width and height. As the probe feature size becomes increasingly smaller, a greater burden is placed upon scanner 110 and associated software such as applications 172 in order to meet certain specification requirements. For example, some embodiments of scanner 110 and applications 172 may require that what may be referred to as the linearity in either the X or Y axes may not be off by more than +/−1 pixel where the pixel size may in some implementations be smaller than +/−1m. The term “linearity” as used herein generally refers to a difference in the positional relationship of a pixel placed in an image and the actual physical position on the array that the pixel represents, i.e. how true is the placement of a pixel in an image to the actual physical position. In the present example, cost efficient hardware components alone may not be able to achieve the specified requirements where the image analysis application may implement one or more methods of correction in order to meet those requirements.

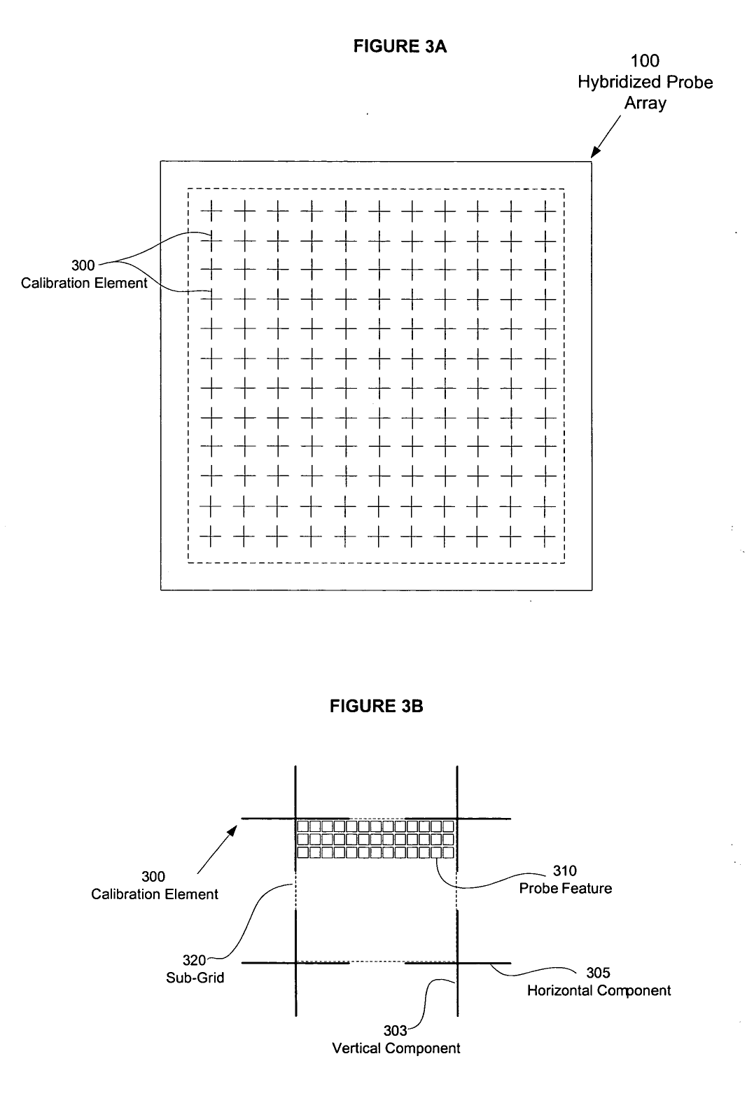

Embodiments of the present invention include one or more calibration elements 300 that may be used in one or more calibration operations of scanner 110. For example, calibration elements 300 may be disposed in the active area of probe array 100 as illustrated in FIG. 3A. In the present example, calibration elements 300 may be composed of a range of materials including one or a combination of materials that reflect more light compared to the substrate of array 100 such as, for instance, chrome elements deposited on the substrate of array 100 by methods known to those in the related art. Alternatively, some embodiments may include calibration elements 300 composed of materials that reflect less light as compared to the substrate of array 100. Also in some embodiments, elements 300 may not be composed of any material but instead be created by the removal of substrate material such as by etching or grinding probe array 100.

As illustrated in FIGS. 3A and 3B, calibration elements 300 may include a rectilinear or other type of pattern such as, for instance, crosshair or “+” shaped elements, where the exact position of one or more components of each of the elements may be exactly defined. The term “rectilinear”, as used herein, generally refers to a pattern that is formed in or generally comprises straight lines. For example, other implementations of elements 300 may include other shapes such as, for instance vertical or horizontal bars where the repeating pattern on array 100 may comprise a ladder-like pattern. In the present example, vertical component 303 of each of a plurality of elements 300 may be employed by applications 399 as a ladder-like pattern for X-axis linearity calibration and horizontal component 305 may be employed for Y-axis calibration. Continuing with the present example, the exact positions of each of the edges of vertical component 303 and horizontal component 305 of each of elements 300 are defined and stored in one or more data files or databases such as, for instance, calibration feature data 530 of calibration data 576. The positions may be defined by spatial position with respect to one or more reference points on array 100 or alternatively one or more reference points outside of array 100 such as, for instance, border elements, active area boundary 210, probe features 310, focus elements 200, or other feature associated with probe array 100 that may be employed as a reference point. In the same or another implementation, one or more of elements 300 may act as reference points for one or more other elements 300.

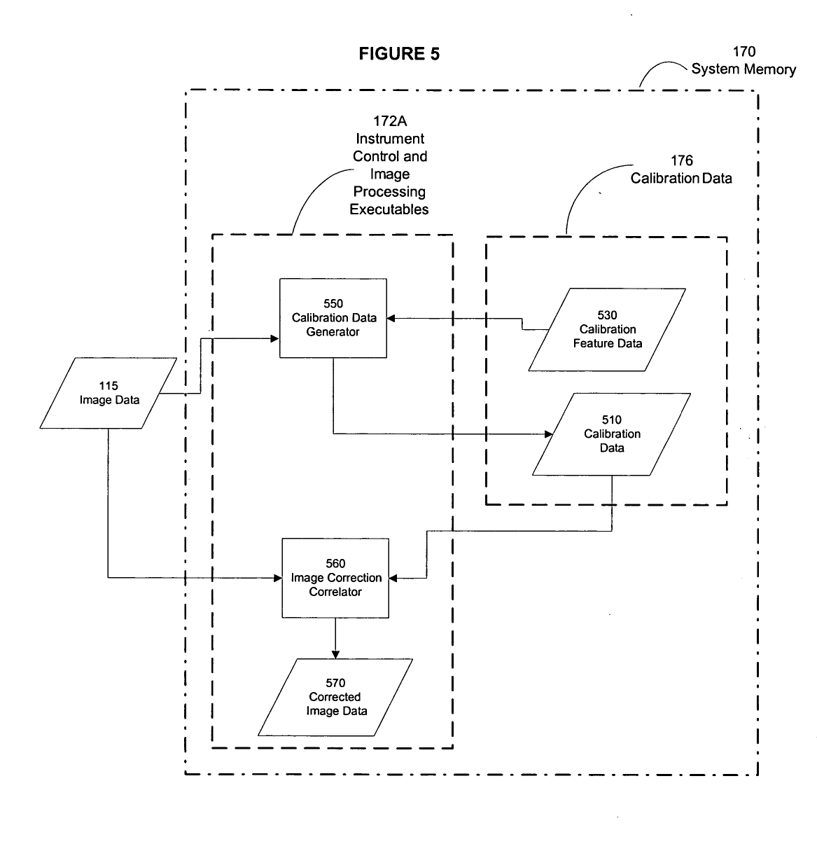

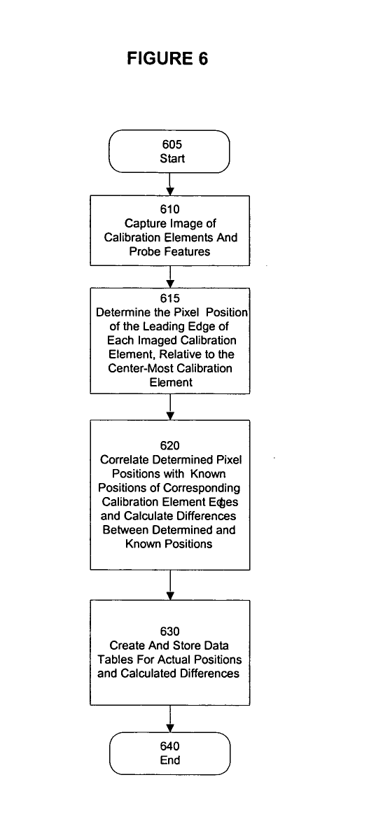

Embodiments of the present invention include applications 399 performing one or more linearity calibration operations as the normal operating procedure of every scan performed by scanner 110. Thus the possibility of linearity errors in the corrected image is significantly reduced due, in part, to an accurate measure of the error present in scanner 110 during the acquisition of the raw image. Illustrative examples of a system and method for generating calibration tables and image correction are presented in FIGS. 5 and 6.

For example, Step 610 illustrates the step where applications executables 172A instructs scanner 110 to collect image data 115 from probe array 100 that includes reflected excitation light from elements 300 and emitted light from a plurality of fluorescent molecules associated with targets hybridized to probe features 310. Calibration data generator 550 receives image data 115 and, as illustrated in step 615 performs an analysis known to those of ordinary skill in the related art that includes identifying the image of each of elements 300 based, at least in part, upon one or more characteristics of detected reflected excitation light. Generator 550 may then determine the position in image data 115 of the leading edge of vertical component 303, horizontal component 305 or both, using one or more measures of scale such as, for instance, the number of pixels away from a reference point or other measure known to those in the related art. In the present example, the reference point may include the calibration element locate at the center position in image data 115 or other selected element 300 that may be identified in calibration feature data 530.

Continuing the example from above, step 620 illustrates the step of generator 550 correlating the determined positions of the leading edges of each of the components of each of elements 300 with the known physical positions of the corresponding leading edge stored in calibration feature data 530 and generating an offset or error correction value for each that is associated with the difference between the determined position and the known position. In some embodiments, generator 550 may then calculate an error correction value for each pixel position located between the vertical and horizontal components of two adjacent elements 300 such as, for instance, for each pixel position associated with each of probe features 310 based, at least in part upon an interpolation of an the error correction values associated with each of the respective components. In the present example, the offset or error correction value may be measured using a number of scales known to those of ordinary skill in the related art, including numbers of pixels, and may be stored as calibration data 510 as illustrated in step 630. Calibration data 510 may include one or more error correction tables such as, for instance, an X-axis linearity error correction table based, at least in part upon error correction value associated with vertical component 303, and a Y-axis linearity error correction table based, at least in part upon error correction value associated with horizontal component 305. Alternatively, data 510 may include a single table with both the X-axis and Y-axis values.

Image correction correlator 560 may then implement one or methods of applying each of the error correction values in data 510 to image data 115 to correct for the measured error. For example, correlator 560 may correct the pixel placement error in image data 115 independently in a plurality of sub-grids that may be defined by all or part of the horizontal and vertical components of calibration elements 300 such as, for instance, sub-grid 320, where the sub-grid is defined by the plane of the vertical and horizontal components of four adjacent elements 300. In the present example, the pixel placement correction in a first sub-grid 320 is not affected by the pixel placement correction in another sub-grid 320.

Continuing the example from above, correlator 560 may systematically interrogate each pixel position present in sub-grid 320 and apply one or more associated error correction values in data 510. In the present example, correlator 560 may apply an X-axis correction and a Y-axis correction for each pixel position interrogated. Also in the present example, each of the X-axis corrections and Y-axis corrections may be implemented relative to one or more reference positions that may include one or more components of elements 300 that define sub-grid 320. Once correlator 560 has corrected each pixel position associated with image data 115, the data may be stored in one or more locations as a separate image file such as corrected image data 570.

Similar to the embodiment described above with respect to auto-focus operations, elements 300 may be associated with a plurality of implementations of probe array 100 disposed on a wafer or in individual wells. For example, executables 172A may perform one or more calibration operations as described above for each implementation of probe array 100, or alternatively with a subset of one or more implementations that may, for instance, provide a representative estimation of error associated with scanner 110 and the plurality of probe arrays 100.

Yet another embodiment of calibration elements 300 may be implemented using CCD or cooled CCD type detectors where an image may be acquired and image correction methods applied based, at least in part upon one or more reference points provided by elements 300 in the image.

Those of ordinary skill in the related art will appreciate that the above examples are for the purpose of illustration only, and that many methods of generating and applying error correction or offset values exist. Also, calibration elements 300 may also be employed for additional methods of calibration such as, for instance, calculating a radius value associated with embodiments of scanner 110 that employ what may be referred to as a galvanometer arm or other means of translating a beam across probe array 100. Example systems and methods for generating and applying error correction or offset values and other calibration procedures that may employ calibration elements 300 are described in U.S. patent application Ser. No. 10/389,194, incorporated by reference above.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

Also, the functions of several elements may, in alternative embodiments, be carried out by fewer, or a single, element. Similarly, in some embodiments, any functional element may perform fewer, or different, operations than those described with respect to the illustrated embodiment. Also, functional elements shown as distinct for purposes of illustration may be incorporated within other functional elements in a particular implementation. Also, the sequencing of functions or portions of functions generally may be altered. Certain functional elements, files, data structures, and so on may be described in the illustrated embodiments as located in system memory of a particular computer. In other embodiments, however, they may be located on, or distributed across, computer systems or other platforms that are co-located and/or remote from each other. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

What is claimed is:

1. A system, for providing focus elements associated with a biological probe array, comprising:
a biological probe array having an active area; and
a plurality of focus elements associated with the biological probe array, wherein the focus elements include an unambiguous pattern.

2. The system of claim 1, wherein:
the plurality of focus elements are disposed outside of the active area.

3. The system of claim 1, wherein:
the plurality of focus elements represent reflective elements.

4. The system of claim 3, wherein:
the reflective elements represent chrome elements.

5. The system of claim 1, wherein:
the plurality of focus elements are enabled to hybridize one or more target molecules.

6. The system of claim 5, wherein:
the one or more target molecules represent molecules present in a biological sample.

7. The system of claim 5, wherein:
the one or more target molecules represent molecules added by a user.

8. The system of claim 1, wherein:
the unambiguous pattern represents a checkerboard pattern.

9. The system of claim 1, further comprising:
a scanner for acquiring an image of the plurality of focus elements; and
an image analysis application enabled to execute one or more positional adjustments of the biological probe array based, at least in part, upon the image of the plurality of focus elements.

10. The system of claim 9, wherein:
the one or more positional adjustments of the biological probe array represents translating the biological probe array in one or more axes, wherein the one or more axes represents a X, Y, Z, roll, and pitch axes.

11. The system of claim 9, wherein:
the one or more positional adjustments represent placing each of the plurality of probes in a best plane of focus.

12. The system of claim 9, wherein:
the image analysis application applies a deconvolution method to the image of the plurality of focus elements.

13. A method, for positional adjustment of a biological probe array, comprising:
acquiring an image of a plurality of focus elements disposed on a biological probe array, wherein a plurality of probes are disposed in an active area of the biological probe array; and
executing one or more positional adjustments of the biological probe array based, at least in part, upon the image of the plurality of focus elements, wherein the plurality of focus elements represent an unambiguous pattern.

14. The method of claim 13, wherein:
the plurality of focus elements are disposed outside of the active area.

15. The method of claim 13, wherein:
the unambiguous pattern represents a checkerboard pattern.

16. The method of claim 13, wherein:
the step of executing one or more positional adjustments represents translating the biological probe array in one or more axes, wherein the one or more axes represents a X, Y Z, roll, and pitch axes.

17. The method of claim 13, wherein:
the one or more positional adjustments represents placing each of the plurality of probes in a best plane of focus.

18. A method, for positional adjustment of a biological probe array, comprising:
acquiring an image of a plurality of focus elements disposed on a biological probe array, wherein a plurality of probes are disposed in an active area of the biological probe array;
applying a deconvolution method to the image of the plurality of focus elements; and
executing one or more positional adjustments of the biological probe array based, at least in part, upon the deconvolution method of the image.

19. The method of claim 18, wherein:
the step of executing one or more positional adjustments represents placing each of the plurality of probes in a best plane of focus.

20. A system, for providing calibration elements associated with a biological probe array, comprising:
a biological probe array having an active area of the biological probe array; and
a plurality of calibration elements disposed on the biological probe array, wherein the plurality of calibration elements are disposed in a rectilinear pattern.

21. The system of claim 20, wherein:
the plurality of calibration elements are disposed in the active are of the biological probe array.

22. The system of claim 20, wherein:
each of the plurality of calibration elements represents a chrome element.

23. The system of claim 22, wherein:
each of the plurality of calibration elements has a vertical component and a horizontal component.

24. The system of claim 23, wherein:
the vertical component is associated with X-axis linearity and the horizontal component is associated with Y-axis linearity.

25. The system of claim 20, further comprising:
a scanner for acquiring an image of a plurality of probes and the plurality of calibration elements; and
an image analysis application enabled to generate a plurality of error correction values.

26. The system of claim 25, wherein:
each error correction value is based, at least in part, upon a difference between a position of each calibration element in the acquired image and an actual position of a corresponding calibration element disposed on the biological probe array.

27. The system of claim 25, wherein:
each of the plurality of error correction values is associated with a pixel position in the image.

28. A method, for determining error associated with a scanner instrument, comprising:
acquiring an image of a plurality of target molecules hybridized to a plurality of probes and a plurality of calibration elements; and
generating a plurality of error correction values, wherein each error correction value is based, at least in part, upon a difference between a position of each calibration element in the acquired image and an actual position of a corresponding calibration element disposed on the biological probe array.

29. The method of claim 28, wherein:
the plurality of calibration elements are disposed in a rectilinear pattern.

30. The system of claim 28, wherein:
the plurality of calibration elements are disposed in the active are of the biological probe array.

31. A method, for determining error associated with a scanner instrument, comprising:
acquiring an image of a plurality of target molecules hybridized to a plurality of probes and a plurality of calibration elements;
generating a plurality of error correction values, wherein each error correction value is based, at least in part, upon a difference between a position of each calibration element in the acquired image and an actual position of a corresponding calibration element disposed on the biological probe array; and
correlating one or more of the plurality of error correction values with each of a plurality of pixels in the image, wherein the correlating represents adjusting the position of each pixel based, at least in part, upon the one or more error correction values.

32. The method of claim 31, wherein:
the one or more error correction values represents an X-axis linearity correction value.

33. The method of claim 31, wherein:
the one or more error correction values represents a Y-axis linearity correction value.

34. The method of claim 31, wherein:
the plurality of pixels in the image represents all pixels in the image.

35. A system, for providing focus elements associated with a plurality biological probe arrays, comprising:
a plurality of biological probe arrays each having an active area; and
a plurality of focus elements disposed on the biological probe array, wherein the focus elements represent an unambiguous pattern.

36. A system, for providing calibration elements associated with a plurality biological probe arrays, comprising:
a plurality of biological probe arrays each having an active area; and
a plurality of calibration elements disposed on the biological probe array, wherein the plurality of calibration elements are disposed in a rectilinear pattern.
